(19) United States
(12) Patent Application Publication
(10) Pub. No.: US 2010/0267994 A1
(43) Pub. Date: Oct. 21, 2010

(54) PROCESSES FOR PREPARING POLYTRIMETHYLENE GLYCOL USING ION EXCHANGE RESINS

(75) Inventor: Rupert SPENCE, Kingston (CA)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E. I. DU PONT DE NEMOURS AND COMPANY, Wilmington, DE (US)

(21) Appl. No.: 12/759,834

(22) Filed: Apr. 14, 2010

Related U.S. Application Data
(60) Provisional application No. 61/169,829, filed on Apr. 16, 2009.

(51) Int. Cl. C07C 41/01

(52) U.S. Cl. 568/671

(57) ABSTRACT

Processes for preparing polytrimethylene ether glycol or copolymers thereof using an acid polycondensation catalyst and ion exchange resins are provided.

### FIELD OF THE INVENTION

The present invention is related to processes for preparing polytrimethylene ether glycol and copolymers thereof.

### BACKGROUND

Polyalkylene ether glycols can be prepared by the acid-catalyzed elimination of water from the corresponding alkylene glycol or the acid-catalyzed ring opening of the alkylene oxide. For example, polytrimethylene ether glycol (PO3G) can be prepared by dehydration of 1,3-propanediol (3G) or by ring-opening polymerization of oxetane using soluble acid catalysts.

Methods for making PO3G from 3G using a sulfuric acid catalyst are disclosed in U.S. Patent Application publication Nos. 200210007043A1 and 200210010374A1. Polyol synthesis conditions largely determine the amounts and types of impurities, color precursors, and color bodies formed, and purification of the PO3G is frequently required before its use in commercial applications. The purification process for polytrimethylene ether glycol typically comprises: (1) a hydrolysis step to hydrolyze the acid esters formed during the polymerization; (2) water extraction steps to remove the acid catalyst, unreacted monomer, low molecular weight linear oligomers and oligomers of cyclic ethers; (3) a base treatment, typically with a slurry of calcium hydroxide, to neutralize and precipitate the residual acid present; and (4) drying and filtration of the polymer to remove the residual water and solids.

When sulfuric acid is used as a catalyst, it is preferred to include a hydrolysis step because a substantial portion of the acid is converted to the ester, an alkyl hydrogen sulfate. These ester groups act as emulsifying agents during the water washing process, causing the washing process to be difficult and time-consuming and also making the acid removal incomplete. The hydrolysis step is also important in order to obtain polymer with the high dihydroxy functionality required to use the polymer as a reactive intermediate. The purification processes disclosed in the prior art are effective in producing polytrimethylene ether glycol with high dihydroxy functionality. Often, however, it is desirable to produce short chain or low molecular weight PO3G from the polycondensation of 1,3-propanediol. As disclosed in U.S. Pat. No. 2,520,733, trimethylene glycol polymers having molecular weights below about 200 are generally water-soluble, and PO3G with molecular weight below about 1,000 contains significant amounts of water-soluble oligomers. In addition to the solubility of oligomers in water, the solubility of water in the low molecular polymer and interactions between polymer and water molecules can make it hard to achieve a distinct aqueous and organic phase separation. Also, the water washing steps remove the acid present but also can remove any water-soluble short polyether chains. In order to achieve high polymer yields, it is essential to recover the soluble fraction of the polymer from the water solutions, a process which can be expensive and time-consuming, requiring distillation of large amounts of water and incurring high capital, maintenance, and operating costs. It would be highly desirable if low molecular weight polytrimethylene ether glycol free of catalyst contamination could be prepared by acid catalyzed polymerization without the need for water washing steps

In U.S. Pat. No. 7,074,969, a process is disclosed for making polytrimethylene ether glycol. The process requires the use of a filter aid and the disclosed “substantially water-insoluble bases” include inorganic bases-metal oxides, metal hydroxides and metal carbonates

It would be desirable to have a process for producing PO3G or copolymers thereof of the desired molecular weight and purity in which the acid catalyst is removed from the reaction mixtures efficiently without the need for a filter aid and in a manner that allows for recycle of the catalyst.

### SUMMARY OF THE INVENTION

One aspect of the present invention is a process for making polytrimethylene ether glycol or copolymers thereof comprising:

(a) polycondensing at least one reactant selected from the group consisting of 1,3-propanediol, oligomers of 1,3-propanediol having a degree of polymerization of 2-6, and mixtures thereof, in the presence of an acid polycondensation catalyst at a temperature of at least about 150° C. to obtain a polytrimethylene ether glycol reaction mixture;

(b) contacting the polytrimethylene ether glycol reaction mixture with a basic ion exchange resin; and

(c) separating the polytrimethylene ether glycol from the basic ion exchange resin to obtain polytrimethylene ether glycol.

### DETAILED DESCRIPTION

Provided herein is a process for making polytrimethylene ether glycol or copolymers thereof comprising: polycondensing at least one reactant selected from the group consisting of 1,3-propanediol, oligomers of 1,3-propanediol having a degree of polymerization of 2-6, and mixtures thereof in the presence of an acid polycondensation catalyst at a temperature of at least about 150° C. to obtain a polytrimethylene ether glycol reaction mixture; contacting the polytrimethylene ether glycol reaction mixture with a basic ion exchange resin; and separating the polytrimethylene ether glycol from the basic ion exchange resin to obtain polytrimethylene ether glycol.

In some embodiments, the contacting with a basic ion exchange resin removes at least about 60% of the acid polycondensation catalyst from the polytrimethylene ether glycol reaction mixture, and, in some embodiments, the reactant comprises 90% or more by weight of 1,3-propanediol. The processes may further comprise the step of removing unreacted reactant by distillation at reduced pressure following the separation step. In some embodiments, the polycondensation step is carried out at a temperature of from about 150° C. to about 210° C.

In some embodiments, the acid polycondensation catalyst for the process is selected from the group consisting of Bronsted acids, Lewis acids and super acids. The acid polycondensation catalyst may be selected from the group consisting of sulfuric acid, hydriodic acid, fluorosulfonic acid, p-toluenesulfonic acid, benzenesulfonic acid, methanesulfonic acid, trifluoromethanesulfonic acid, 1,1,2,2-tetrafluoroethanesulfonic acid, and 1,1,1,2,3,3-hexafluoropropanesulfonic acid. The acid polycondensation catalyst may be used in an amount of from about 0.1 wt % to about 1 wt % based on the weight of the reactants. In some embodiments, the acid polycondensation catalyst is triflic acid.

In some embodiments, the basic ion-exchange resin is selected from the group consisting of quarternary ammonium type or tertiary amine type, and, in some embodiments, the contacting and the separation comprise filtering the reaction mixture through a column of ion exchange resin. In some embodiments, the polytrimethylene ether glycol contains from about 0 to about 10 ppm of sulfur.

In some embodiments of the processes provided herein, the polytrimethylene ether glycol has a molecular weight of from about 200 to about 5,000, from about 250 to about 750, or from about 200 to about 1000.

By contacting the polytrimethylene ether glycol reaction mixture with a basic ion exchange resin, the acid catalyst is removed. In some embodiments at least about 60%, at least about 70%, at least about 80% or at least about 90% of the acid catalyst is removed.

In some embodiments, the process further comprises removing volatile unreacted reactants or by-products by distillation at reduced pressure following the separation step (c) above.

Also provided is a process for manufacture of polytrimethylene ether glycol using an acid polycondensation catalyst. The process can be employed for manufacture of low molecular weight polytrimethylene ether glycol. The starting material for the process is at least one reactant selected from the group consisting of 1,3-propanediol, oligomers of 1,3-propanediol having a degree of polymerization of 2-6, and mixtures thereof. The 1,3-propanediol reactant employed in the processes disclosed herein can be obtained by any of the various chemical routes or by biochemical transformation routes. Suitable routes are disclosed in U.S. Pat. Nos. 5,015,789, 5,276,201, 5,284,979, 5,334,778, 5,364,984, 5,364,987, 5,633,362, 5,686,276, 5,821,092, 5,962,745, 6,140,543, 6,232,511, 6,235,948, 6,277,289, 6,284,930, 6,297,408, 6,331,264 and 6,342,646. In some embodiments, the 1,3-propanediol used as the reactant or as a component of the reactant has a purity of greater than about 99% by weight as determined by gas chromatographic analysis.

In some embodiments 1,3-propanediol, dimers and/or trimers of 1,3-propanediol are used as the reactant. In other embodiments, the reactant comprises about 90% or more by weight of 1,3-propanediol. In one embodiment, the reactant comprises 99% or more by weight of 1,3-propanediol.

In some embodiments, the process further comprises including in the polycondensing step at least one comonomer diol reactant selected from the group consisting of ethylene glycol, C₄-C₁₂ straight-chain diols, and C₃-C₁₂ branched diols. The total reactant may contain up to about 20 wt % of comonomer diols, in addition to the reactant 1,3-propanediol or its dimers and trimers. Examples of suitable comonomer diols include ethylene glycol, 2-methyl-1,3-propanediol, 2,2-dimethyl-1,3-propane diol, and C₆-C₁₂ diols such as 2,2-diethyl-1,3-propane diol, 2-ethyl-2-(hydroxymethyl)-1,3-propane diol, 1,6-hexanediol, 1,8-octanediol, 1,10-decanediol, 1,12-dodecanediol, 1,4-cyclohexanediol, and 1,4-cyclohexanedimethanol. In another embodiment, the comonomer diol is ethylene glycol. Poly(trimethylene-ethylene ether) glycols prepared from 1,3-propanediol and ethylene glycol are disclosed in U.S. Patent Application Publication No. 2004/0030095. In one embodiment, the starting material for the process is at least one reactant selected from the group consisting of 1,3-propanediol, oligomers of 1,3-propanediol having a degree of polymerization of 2-6, and mixtures thereof and at least one comonomer diol. In one embodiment, the comonomer diol is ethylene glycol.

The polycondensation can be carried out as a batch, semi-continuous, or continuous process. Generally, the polytrimethylene ether glycol is prepared by a process comprising the steps of: (a) providing (1) reactant, and (2) acid polycondensation catalyst; and (b) polycondensing the reactants to form a polytrimethylene ether glycol. In one embodiment, the reaction is conducted at an elevated temperature of at least about 150° C. In another embodiment, the reaction is conducted at an elevated temperature of at least about 160° C. up to about 210° C. In some embodiments, the reaction is conducted at an elevated temperature of at least about 160° C. up to about 200° C. In some embodiments, the reaction is conducted at a temperature of at least about 150° C. up to about 250° C.

Polytrimethylene ether glycol in accordance with the processes disclosed herein can be prepared by a continuous process comprising: (a) continuously providing (i) reactant, and (ii) polycondensation catalyst; and (b) continuously polycondensing the reactant to form polytrimethylene ether glycol. The polycondensing can be carried out in two or more reaction stages. The polytrimethylene ether glycol can be prepared at atmospheric pressure or below. In some embodiments, the pressure is less than 500 mm Hg, or less than 250 mm HG. In other embodiments, still lower pressures, even as low as 1 mm Hg can be used, such as for small scale operation, for example, and for larger scale, pressure is at least 20 mm Hg, preferably at least 50 mm Hg. On a commercial scale, in some embodiments, the polycondensation pressure may be between 50 and 250 mm Hg. In some embodiments, the polycondensation is performed at a temperature of less than about 250° C., less than about 220° C. or less than about 210° C. In some embodiments, the polycondensing is carried out at temperatures greater than about 150° C., greater than about 160° C., or greater than about 180° C.

In one embodiment, the polycondensation is carried out in an up-flow co-current column reactor and the reactant, and polytrimethylene ether glycol flow upward co-currently with the flow of gases and vapors. The reactor has 3-30 stages. The reactant can be fed to the reactor at one or multiple locations. In another embodiment, the polycondensation is carried out in a counter current vertical reactor wherein the reactant and polytrimethylene ether glycol flow in a manner counter-current to the flow of gases and vapors. In such a process, the reactor has two or more stages. Typically, the reactant is fed at the top of the reactor.

The reaction time for either batch or continuous polycondensation will depend on the polymer molecular weight that is desired and the reaction temperature, with longer reaction times producing higher molecular weights. In one embodiment, the reaction times are from about 1 hour to about 20 hours. In another embodiment, the reaction times are from about 1 hour to about 50 hours. In other embodiments, reaction times may be from about 5 hours to about 20 hours or from about 10 hours to about 20 hours or from about 10 hours to about 40 hours.

Any acid catalyst suitable for acid catalyzed polycondensations of 1,3-propanediol may be used in present process. Certain useful acid polycondensation catalysts are disclosed in U.S. Published Patent Application Nos. 2002/0007043 A1 and in U.S. Pat. No. 6,720,459. Suitable acid catalysts include homogeneous Lewis acids, Bronsted acids, super acids, and mixtures thereof. In one embodiment, the catalysts are selected from the group consisting of inorganic acids, organic sulfonic acids, heteropolyacids and metal salts. In one embodiment, the catalyst is a homogeneous catalyst selected from the group consisting of sulfuric acid, hydriodic acid, fluorosulfonic acid, phosphorous acid, p-toluenesulfonic acid, benzenesulfonic acid, methanesulfonic acid, phosphotungstic acid, trifluoromethanesulfonic acid (triflic acid), phosphomolybdic acid, 1,1,2,2-tetrafluoro-ethanesulfonic acid, and 1,1,1,2,3,3-hexafluoropropanesulfonic acid, bismuth triflate, yttrium triflate, ytterbium triflate, neodymium triflate, lanthanum triflate, scandium triflate, and zirconium triflate. In one embodiment, the catalyst is triflic acid.

Homogeneous catalysts can also include rare earth acids of the form La(1,1,2,2,-tetrafluoroethane sulfonate)₃, La(1,1,2,3,3,3-hexafluoropropanesulfonates)₃, Sc(1,1,2,2,-tetrafluoroethane sulfonate)₃, Sc(1,1,2,3,3,3-hexafluoropropanesulfonates)₃, Ac(1,1,2,2,-tetrafluoroethane sulfonate)₃, Ac(1,1,2,3,3,3-hexafluoropropanesulfonates)₃, Yb(1,1,2,2,-tetrafluoroethane sulfonate)₃ and Yb(1,1,2,3,3,3-hexafluoropropanesulfonates)₃, as well as SbF₅—HF (magic acid) and mixtures of fluorosulfuric acid and antimony pentachloride, as disclosed by G. A. Olah, G. K. Surya Prakash and J. Sommer in “Superacids” (John Wiley & Sons, NY, 1985).

The acid polycondensation catalyst is typically used in an amount of from about 0.01 wt % to about 3 wt %, or from about 0.05 wt % up to about 2 wt %, or from about 0.1 wt % to about 0.5 wt %, based on the weight of the reactants.

Contacting the polycondensation reaction mixture with a basic ion exchange resin enables the removal of acid catalyst residues without the need for a filter aid. The acid catalyst residues can be removed across a broad molecular weight range of PO3G, including for low molecular weight PO3G, without substantial yield loss and without changes in polymer properties.

The resin can be added as a dry solid, or as an aqueous slurry. Suitable basic ion exchange resins include, for example, strongly basic resins (e.g. quaternary ammonium type) or weakly basic resins (e.g. tertiary amine type) from Dow Chemicals (e.g. Dowex brand) and Rohm and Haas (e.g. Amberlyst brand).

The contacting of the polytrimethylene ether glycol is carried out at a temperature of at least about 25° C. to about 150° C.

In one embodiment, the amount of resin used in the contact step is at least enough to neutralize all of the acid polycondensation catalyst. In one embodiment, an excess of from about 0.1 wt. % to about 10 wt. % is used. in some embodiments, at least about 60%, at least about 70%, at least about 80% or at least about 90% of the acid catalyst will be removed. Although it is envisioned that the processes disclosed herein can be used to remove the acid catalyst such that no other steps to remove catalyst are necessary, it is contemplated that a portion of the acid catalyst may be removed using the processes herein and that other purification methods are also employed.

In one embodiment, the ion exchange resin is used in a fixed bed column and the contacting of step (b) and the separation of step (c) comprise filtering the reaction mixture through a column of ion exchange resin. Alternatively, the ion exchange resin is added to the polytrimethylene ether glycol reaction mixture, and then removed by filtration or other conventional solid-liquid separation processes. The period of contact between the reaction mixture and the ion exchange resin may be at least about 1 minute up to about 10 hours. In one embodiment, the treatment with ion exchange resins is performed under an inert atmosphere to avoid discoloration of the PO3G.

After the treatment step, the resins can be recycled and reused by washing the resin with an aqueous basic solution. Recycling of ion exchange resins is a common practice and known to those skilled in the art. The acid catalyst can also be recovered for re-use as is known in the art. The ability to recover the acid and the ion exchange resin can reduce manufacturing cost of PO3G and provide a more environmentally friendly process.

The processes disclosed herein provide a high purity polytrimethylene ether glycol having a number average molecular weight greater than about 200 and less than about 5,000. One advantage of the processes is that they can be used to produce low molecular weight polytrimethylene ether glycol, i.e. having a number average molecular weight from about 200 to about 1,000, without significant loss of the water-soluble or water sensitive oligomer fraction during the acid polycondensation catalyst removal step. In one embodiment, the polytrimethylene ether glycol has a number average molecular weight of about 200 to about 5,000. In one embodiment, the polytrimethylene ether glycol product has a molecular weight of about 250 to about 750.

The products produced by the processes disclosed herein preferably have a color of less than about 100 APHA, more preferably about 50 APHA or less, and end group unsaturation less than about 15 meq/kg. The color of the products can be further improved, if desired, by the method disclosed in U.S. patent application US 2004-0225162 A1. Thermal stabilizers, antioxidants and/or coloring materials can be added to the polymerization mixture or final product.

Unless stated otherwise, all percentages, parts, ratios, etc., are by weight. Trademarks are shown in upper case. Further, when an amount, concentration, or other value or parameter is given as either a range, preferred range or a list of upper preferable values and lower preferable values, it is intended to include all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Some embodiments of the invention are illustrated in the following examples.

### EXAMPLES

The 1,3-propanediol utilized in the examples was prepared by biological methods and had a purity of >99.8%.

The number-average molecular weights (Mn) were determined by end-group analysis using NMR spectroscopic methods. Fluorine content was measured by Neutron Activation Analysis.

Color was measured as APHA values (Platinum-Cobalt System) according to ASTM D-1209.

Unsaturation was determined by NMR.

Ion exchange resin XUS 43568.00 for acid removal from lubricants (from Dow Chemical) is a weak base anion with a styrene-DVB, macroporous matrix and a tertiary amine functional group.

DOWEX M43 ion exchange resin (from Dow Chemical) is a weak base anion with a styrene-DVB, macroporous matrix and a tertiary amine used in corrosion control applications.

Amberlyst A26OH resin (from Rohm&Haas) is an industrial grade strong base polymeric resin with a macroreticular matrix shipped as a hydroxide form.

### Example 1

### Preparation of PO3G with Mn=˜3000 Using Triflic Acid

A 22 L glass reactor equipped with a mechanical stirrer, a nitrogen dip tube tipped with a glass fritted sparger and an over head condenser unit was sparged with N₂. To the reactor was added 12 kg of 1,3-propanediol and 12 g (0.1 wt %) of triflic acid. The reaction mixture was then heated (using a 120V heating mantel) to 180° C. while sparging with nitrogen (3 L/min) and mixing at 250 rpm. After 13 hours, the nitrogen sparging rate was increased to 10 L/min and water addition was started at a rate of 1.5 ml/min to the reaction via a small pump connected to the nitrogen addition tube. After 17 hours, the Mn of the polymer reaction mixture was 303 and the moisture 3461 ppm. At this time, the reaction temperature was decreased to 165° C. At 19 hours, the water injection was decreased to 1 ml/min. After 26 hours, the Mn of the polymer reaction mixture was 942 and the moisture 2297 ppm. At this time, the reaction temperature was decreased to 155° C. The reaction was maintained at 155° C. until the end of the experiment. The reaction was shut down at 51.5 hours by setting the condenser head to reflux, decreasing the temperature of the heating mantel, and increasing the water injection to 5 ml/min for 20-30 minute. The final polymer had a Mn=2821, unsaturates=16 meq/kg, an APHA=14 and F content ˜500 ppm. The only source of fluorine in the polymer is the fluorinated groups in the acid, thus, the fluorine content is an indication of residual acid.

### Example 2

### Preparation of PO3G with Mn=˜500 Using Triflic Acid

In a 50 gallon glass-lined, baffled, oil-heated reactor, 120 kg of 1,3-propanediol and 0.1 wt % of triflic acid were combined under nitrogen. The reaction mixture was heated to 185° C. while sparging with nitrogen (30 L/min) and mixing at 120 rpm. After 10.5 hours, the Mn of the polymer reaction mixture was 254 and the moisture 6100 ppm. At this time, water addition to the reaction mixture was started at a rate of 30 ml/min and the nitrogen sparge rate was increased to 100 L/min. The reaction was shut down at 13 hours by stopping the nitrogen flow, decreasing the temperature of the oil skid to 95° C., and by adding several kilos water into the reactor. The final polymer had a Mn=481, unsaturates=25 meq/kg, an APHA=26, and F concentration=546 ppm.

### Example 3

### Preparation of PO3G with Mn=˜3000 Using TFESA (1,1,2,2-tetrafluoro-ethanesulfonic acid)

A 22 L glass reactor equipped with a mechanical stirrer, a nitrogen dip tube tipped with a glass fritted sparger and an overhead condenser unit was sparged with N₂. To the reactor was added 12 kg of 1,3-propanediol and 15 g (0.125 wt %) of TFESA acid. The reaction mixture was then heated (using a 120 V heating mantel) to 180° C. while sparging with nitrogen (3 L/min) and mixing at 250 rpm. After 18 hours, the nitrogen sparging rate was increased to 10 L/min and water addition to the reaction was started at a rate of 1 ml/min via a small pump connected to the nitrogen addition tube. After 19.5 hours, the Mn of the polymer reaction mixture was 430 and the moisture 2468 ppm. At this time, the reaction temperature was decreased to 165° C. After 25 hours, the Mn of the polymer reaction mixture was 870 and the moisture 1367 ppm. At this time, the reaction temperature was decreased to 155° C. After 44 hours, the reaction temperature was decreased to 150° C. The reaction was maintained at 150° C. until the end of the experiment. The reaction was shut down at 48 hours by setting the condenser head to reflux, decreasing the temperature of the heating mantel, and increasing the water injection to 5 ml/min for 20-30 minute. The final polymer had a Mn=2924, unsaturates=15 meq/kg and F content ˜650 ppm.

### Example 4

Acid Catalyst Removal from PO3G Mn=˜3000

The product from Example 1 was used to conduct 4 experiments. Samples of the product (˜200 g) were heated to 65° C. or 95° C. and then a known quantity (2 or 3 wt %) of ion exchange resin XUS 43568.00 (from Dow Chemical Company, Midland, Mich.) was added to the polyol. The reaction mixture was stirred and small samples were removed after 15, 30, 60, 120 and 240 minutes. After filtration, the samples were analyzed by neutron activation to determine the residual fluorine content in the PO3G-containing liquid. The results from the four experiments are shown in Table 1. The results show that the triflic acid is removed from the PO3G on stirring with the ion exchange resin.

TABLE 1
Residual Fluorine Content after Treatment of Crude PO3G
(Mn = ~3000) with XUS 43568.00 (Dow Chemical Co.)

| | 3 wt % XUS | 2 wt % XUS | 3 wt % XUS | 2 wt % XUS |
|---|---|---|---|---|
| | 43568.00 resin | 43568.00 resin | 43568.00 resin | 43568.00 resin |
| Time | @ 95° C. | @ 95° C. | @ 65° C. | @ 65° C. |
| (min) | F (ppm) by Neutron Activation | | | |
| 15 | 195 | 300 | 317 | 361 |
| 30 | 101 | 201 | 218 | 344 |
| 60 | 10 | 90 | 131 | 260 |
| 120 | <4 | 23 | 19 | 159 |
| 240 | <3 | 15 | <5 | 67 |

### Example 5

### Acid Catalyst Removal from PO3G Mn=˜500

The crude product from Example 2 was used to conduct 4 experiments. Samples of the product (˜200 g) were heated to 65° C. or 95° C. and then a known quantity (2 or 3 wt %) of ion exchange resin XUS 43568.00 (from Dow Chemical Company) was added. The reaction mixture was stirred and small samples of the PO3G were removed after 15, 30, 60, 120 and 240 minutes. The samples were filtered and then analyzed by neutron activation to determine the residual fluorine content in the PO3G-containing liquid. The results from the four experiments are shown in Table 2. The results show that the triflic acid is removed from the PO3G on stirring with the ion exchange resin.

TABLE 2
Residual Fluorine Content after Treatment of Crude PO3G
(Mn = ~500) with XUS 43568.00 (Dow Chemical Co.)

| | 3 wt % XUS | 2 wt % XUS | 3 wt % XUS | 2 wt % XUS |
|---|---|---|---|---|
| | 43568.00 | 43568.00 | 43568.00 | 43568.00 |
| Time | resin @ 95° C. | resin @ 95° C. | resin @ 65° C. | resin @ 65° C. |
| (min) | F (ppm) by Neutron Activation | | | |
| 15 | 220 | 322 | 352 | 426 |
| 30 | 119 | 258 | 287 | 359 |
| 60 | 73 | 168 | 200 | 289 |
| 120 | 52 | 100 | 106 | 224 |
| 240 | 35 | 68 | 56 | 140 |

### Example 6

### Acid Catalyst Removal from PO3G Mn=˜3000

The crude product from Example 3 (−150 g) was heated to 95° C. and then 4 wt % of ion exchange resin XUS 43568.00 (from Dow Chemical company) was added. The reaction mixture was stirred and a small sample of the PO3G was removed after 120 minutes. The sample was filtered and analyzed by neutron activation. The residual F content in the PO3G-containing liquid was 5 ppm. The result shows that the TFESA is removed from the PO3G on stirring with the ion exchange resin.

### Example 7

### Acid Catalyst Removal from PO3G Mn=˜500

Samples of product from Example 2 (˜200 g) were heated to 95° C. and then 4 wt % of ion exchange resin M43 (from Dow Chemical Company) or Amberlyst A26OH (from Rohm and Haas, Philadelphia) was added. The reaction mixtures were stirred and small samples of the PO3G were removed after 120 minutes. The samples were filtered and analyzed by neutron activation to determine the residual fluorine content in the PO3G-containing liquid. The results from the experiments are shown in Table 3. The results show that the acid is removed from the PO3G with the M43 and Amberlyst A26OH ion exchange resins.

TABLE 3

| | | |
|---|---|---|
| | | 4 wt % |
| | 4 wt % M40 | Amberlyst A26OH |
| Time | resin @ 95° C. | resin @ 95° C. |

| | |
|---|---|
| (min) | F (ppm) by Neutron Activation |

| | | |
|---|---|---|
| 0 | 546 | 546 |
| 120 | 6 | — |
| 240 | — | <1 |

### Example 8

### Preparation of PO3G Mn=˜2300 and Treatment with XUS 43568.00

A 22 L glass reactor equipped with a mechanical stirrer, a nitrogen dip tube tipped with a glass fritted sparger and an overhead condenser unit was sparged with N₂. To the reactor was added 12 kg of 1,3-propanediol and 12 g (0.1 wt %) of triflic acid. The reaction was then heated (using a 120 V heating mantel) to 180° C. while sparging with nitrogen (3 L/min) and mixing at 250 rpm. After 17 hours, the nitrogen sparging rate was increased to 10 L/min and water addition to the reaction was started at a rate of 1 ml/min via a small pump connected to the nitrogen addition tube. After 19.5 hours, the Mn of the polymer reaction mixture was 400 and the moisture 3872 ppm. At this time, the reaction temperature was decreased to 165° C. After 25 hours, the Mn of the polymer reaction mixture was 931 and the moisture 1285 ppm. At this time, the reaction temperature was decreased to 155° C. The reaction was maintained at 155° C. until the end of the polymerization. The polymerization was terminated at 37 hours by setting the condenser head to reflux, decreasing the temperature of the heating mantel, and increasing the water injection to 5 ml/min for 20-30 minute. The polymer after polymerization had an Mn=2302, unsaturates=15 meq/kg, an APHA=15, and F content=513 ppm.

The reaction mixture was allowed to cooled to 85° C., and 320 g of ion exchange resin XUS 43568.00 was then added. The reaction was heated to 95° C. while sparging with nitrogen (5 L/min) and mixing at 200 rpm. After 3.5 hours, the reaction mixture was filtered hot through a 75 micron wire mesh screen. The filtered PO3G had an Mn=2307, unsaturates=15 meq/kg, an APHA=14 and the F content was found to be <1 ppm.

### Example 9

### Preparation of PO3G Mn=˜500 and Treatment with XUS 43568.00

A 22 L glass reactor equipped with a mechanical stirrer, a nitrogen dip tube tipped with a glass fritted sparger and an overhead condenser unit was sparged with N₂. To the reactor was added 12 kg of 1,3-propanediol and 12 g (0.1 wt %) of triflic acid. The reaction mixture was then heated (using a 120 V heating mantel) to 185° C. while sparging with nitrogen (3 L/min) and mixing at 250 rpm. After 14.5 hours, the nitrogen sparging rate was increased to 10 L/min and water addition to the reaction was started at a rate of 3 ml/min via a small pump connected to the nitrogen addition tube. The reaction was maintained at 185° C. until the end of the polymerization. The polymerization was terminated at 16 hours by setting the condenser head to reflux, decreasing the temperature of the heating mantel, and increasing the water injection to 5 ml/min for 20-30 minutes. The polymer after polymerization had an Mn=508, unsaturates=15 meq/kg, and F content=486 ppm.

The reaction mixture was allowed to cool to 100° C., and then 160 g of ion exchange resin XUS 43568.00 was added. The reaction was heated to 105° C. while sparging with nitrogen (5 L/min) and mixing at 200 rpm. After 1 hour, another 160 g of ion exchange resin XUS 43568.00 was added. After 22 hours, the reaction mixture was filtered hot through 75 micron wire mesh screen. The filtered PO3G had an Mn=537, unsaturates=15 meq/kg, an APHA=20 and the F content was found to be 3 ppm.

### Example 10

### Preparation of PO3G Mn=˜1400 and Treatment with XUS 43568.00

A charge of 120 kg of 1,3-propanediol and 120 g of triflic acid was combined under nitrogen in a 50 gallon, glass-lined, baffled, oil-heated reactor. The reaction was then heated to 180° C. while sparging with nitrogen (30 L/min) and mixing at 120 rpm. After 10 hours, the nitrogen sparging rate was increased to 80 L/min and water addition to the reaction mixture was started at a rate of 20 ml/min. After 16.3 hours, the Mn of the reaction mixture was 408. At this time, the reaction temperature was decreased to 165° C. The reaction was maintained at 165° C. until the end of the polymerization. The polymerization was terminated at 26.6 hours by lowering the temperature of the heating oil and adding several kilos water into the reactor. The polymer after polymerization had a Mn=1397, unsaturates=15 meq/kg, and F content=521 ppm.

The reaction mixture was allowed to cooled to 90-100° C. and then 2.8 kg of ion exchange resin XUS 43568.00 was added. The reaction was heated to 95° C. while sparging with nitrogen (40 L/min) and mixing at 200 rpm. After 3 hours, the reaction mixture was circulated through a 75 micron wire mesh screen filter assembly. The filter containing the used XUS resin was then removed and the PO3G dried by heating at 105° C. under a 80 L/min nitrogen flow. The product was discharged via a small filter to a product drum. The final PO3G had an Mn=1417, unsaturates=16 meq/kg, an APHA=15 and the F content in was found to be <1 ppm.

What we claim is:

1. A process for making polytrimethylene ether glycol or copolymers thereof comprising:
(a) polycondensing at least one reactant selected from the group consisting of 1,3-propanediol, oligomers of 1,3-propanediol having a degree of polymerization of 2-6, and mixtures thereof, in the presence of an acid polycondensation catalyst at a temperature of at least about 150° C. to obtain a polytrimethylene ether glycol reaction mixture;
(b) contacting the polytrimethylene ether glycol reaction mixture with a basic ion exchange resin; and
(c) separating the polytrimethylene ether glycol from the basic ion exchange resin to obtain polytrimethylene ether glycol.

2. The process of claim 1, wherein the contacting with a basic ion exchange resin removes at least about 60% of the acid polycondensation catalyst from the polytrimethylene ether glycol reaction mixture.

3. The process of claim 1, wherein the reactant in step (a) comprises 90% or more by weight of 1,3-propanediol.

4. The process of claim 1, further comprising the step of removing unreacted reactant by distillation at reduced pressure following the separation step (c).

5. The process of claim 1, wherein the polycondensation step (a) is carried out at a temperature of from about 150° C. to about 210° C.

6. The process of claim 1, wherein the acid polycondensation catalyst is selected from the group consisting of Bronsted acids, Lewis acids and super acids.

7. The process of claim 1, wherein the acid polycondensation catalyst is selected from the group consisting of sulfuric acid, hydriodic acid, fluorosulfonic acid, p-toluenesulfonic acid, benzenesulfonic acid, methanesulfonic acid, trifluoromethanesulfonic acid, 1,1,2,2-tetrafluoroethanesulfonic acid, and 1,1,1,2,3,3-hexafluoropropanesulfonic acid.

8. The process of claim 1, wherein the acid polycondensation catalyst is used in an amount of from about 0.1 wt % to about 1 wt % based on the weight of the reactants.

9. The process of claim 7, wherein the acid polycondensation catalyst is triflic acid.

10. The process of claim 1, wherein the basic ion-exchange resin is selected from the group consisting of quarternary ammonium type or tertiary amine type.

11. The process of claim 1, wherein the contacting of step (b) and the separation of step (c) comprise filtering the reaction mixture through a column of ion exchange resin.

12. The process of claim 1, wherein the polytrimethylene ether glycol contains from about 0 to about 10 ppm of sulfur.

13. The process of claim 1, wherein the polytrimethylene ether glycol has a molecular weight of from about 200 to about 5,000.

14. The process of claim 1, wherein the polytrimethylene ether glycol has a molecular weight of from about 250 to about 750.

15. The process of claim 1, wherein the polytrimethylene ether glycol has a molecular weight of from about 200 to about 1000.
